(19) United States
(12) Patent Application Publication
(10) Pub. No.: US 2021/0155976 A1
(43) Pub. Date: May 27, 2021

(54) HYBRIDIZING ALL-LNA OLIGONUCLEOTIDES

(71) Applicant: Roche Diagnostics Operations, Inc., Indianapolis, IN (US)

(72) Inventors: Frank Bergmann, Iffeldorf (DE); Dieter Heindl, Muenchen (DE); Michael Schraeml, Penzberg (DE); Johannes Stoeckel, Muenchen (DE)

(21) Appl. No.: 17/126,439

(22) Filed: Dec. 18, 2020

Related U.S. Application Data
(63) Continuation of application No. PCT/EP2019/066132, filed on Jun. 19, 2019.

(30) Foreign Application Priority Data
Jun. 21, 2018 (EP) 18178946.2

(51) Int. Cl. C12Q 1/6816

(52) U.S. Cl. CPC C12Q 1/6816; C12Q 2527/119; C12Q 2527/101; C12Q 2537/113; C12Q 2525/117; C12Q 2525/204; C12Q 2527/137

(57) ABSTRACT

The present report relates to hybridizing single-stranded (ss-) oligonucleotides which entirely consist of locked nucleic acid (LNA) monomers. The present document shows hybridization experiments with pairs of entirely complementary ss-oligonucleotides which fail to form a duplex within a given time interval. The present report provides methods to identify such incompatible oligonucleotide pairs. In another aspect, the present report provides pairs of complementary ss-oligonucleotides which are capable of rapid duplex formation. The present report also provides methods to identify and select compatible oligonucleotide pairs. In yet another aspect the present report provides use of compatible oligonucleotide pairs as binding partners in binding assays, e.g. immunoassays.

### CROSS-REFERENCE TO RELATED APPLICATIONS

This application is a Continuation of and claims priority to International Patent Application No. PCT/EP2019/066132 (published as WO 2019/243391), filed on Jun. 19, 2019, which claims priority to EP Patent Application No. 18178946.2, filed on Jun. 21, 2018, each of which is hereby incorporated by reference in its entirety.

### INCORPORATION OF SEQUENCE LISTING

A paper copy of the Sequence Listing and a computer readable form of the Sequence Listing containing the file named “P34851_ST25.txt”, which is 1,833 bytes in size as measured in MICROSOFT WINDOWS EXPLORER®, are provided herein and are herein incorporated by reference. This Sequence Listing consists of SEQ ID NOs:1-8.

The present report relates to hybridizing single-stranded (ss-) oligonucleotides which entirely consist of locked nucleic acid (LNA) monomers. The present document shows hybridization experiments with pairs of entirely complementary ss-oligonucleotides which, unexpectedly, fail to form a duplex within a given time interval. The present report provides efficient methods to identify such incompatible oligonucleotide pairs. In another aspect, the present report provides pairs of complementary single-stranded oligonucleotides which entirely consist of locked nucleic acid (LNA) monomers which are capable of rapid duplex formation, surprisingly in the absence of prior denaturation. The present report also provides methods to identify and select such compatible all-LNA ss oligonucleotide pairs. In yet another aspect the present report provides use of compatible oligonucleotide pairs as binding partners in biochemical assays, e.g. binding assays, immunoassays. Specific embodiments are discussed in which compatible LNA oligonucleotide pairs are employed for immobilizing different target molecules, e.g. an analyte-specific capture molecule, in an assay to detect or determine an analyte in a sample.

### BACKGROUND OF THE INVENTION

Particular focus is directed to general biochemical applications in which the specific interaction of the two partners of a binding pair and their eventual connection with each other has a functional role. Very frequently, in heterogeneous immunoassays the biotin:(strept)avidin binding pair is used to immobilize an analyte-specific capture receptor to a solid phase. The present report conceptualizes, explains and demonstrates alternative binding pairs which are suitable for immunoassays, among other applications. Specifically, an alternative binding pair made of two single-stranded all-LNA oligonucleotides capable of forming a duplex by way of hybridization provides a technical alternative to the biotin:(strept)avidin binding pair.

A key focus of the present disclosure is the means with which in the course of an immunoassay the capture receptor is anchored on the solid phase. In particular, the present disclosure focuses on a binding pair which facilitates immobilization of a capture receptor in the presence of a sample containing the analyte, and/or which is capable of anchoring a detection complex after the complex has formed. A binding pair in an immunoassay is technically required to have specific features. Thus, the interaction of the two binding partners has to be specific. Furthermore, the kinetics of connection forming, that is to say the speed with which the two separate partners of the binding pair interact and eventually associate, i.e. bind to each other, is desired to be high. In addition, the connection of the two binding partners is desired to be stable once formed. Moreover, the binding partners must be amenable to chemical conjugation with other molecules such as analyte-specific receptors and solid phase surfaces, for their application in immunoassays. It is important to appreciate that in immunoassays receptors and typically also the analytes to be detected retain their conformation and function only under certain conditions which may differ depending on the particular receptor or analyte that is under specific consideration; thus, a receptor molecule or an analyte may tolerate only limited deviation from these conditions. Such conditions may comprise (but are not limited to) a buffered aqueous solution with a pH in the range of about pH 6 to about pH 8, one or more dissolved salts, one or more helper substances, a total amount of solutes from about 250 to about 400 mosm/kg, at a pre-selected temperature in the range of 20° C. to 40° C., to name but a few.

The separate partners of a binding pair are required to be amenable to conjugation, specifically conjugation with capture molecules i.e. receptors, and conjugation with solid phase surfaces, without losing their ability to specifically associate with, and bind, each other. With regards to conjugates in immunoassays each separate binding partner of the alternative binding pair must be functional under the assay conditions. The same reasoning applies to all other desired materials for conjugation with a binding partner, such as, but not limited to, an analyte, a carrier material, a solid phase, and other substances or compounds that may be present during the course of an assay.

Single-stranded oligonucleotides with complementary sequences, i.e. oligonucleotides capable of forming a duplex by way of hybridization have been proposed earlier as binding pair means to connect macromolecules, or to attach molecules to a solid phase. EP 0488152 discloses a heterogeneous immunoassay with a solid phase on which an analyte-specific capture antibody is immobilized by a nucleic acid duplex which connects the antibody and the solid phase. An embodiment is shown where one hybridized oligonucleotide is attached to the antibody and the complementary oligonucleotide is attached to the solid phase, thereby forming a connecting duplex. Similar disclosures are provided in the documents EP 0698792, WO 1995/024649, WO 1998/029736, and EP 0905517. WO 2013/188756 discloses methods of flow cytometry and a composition comprising an antibody conjugated to a first oligonucleotide, an oligosphere conjugated to a second oligonucleotide having a sequence identical to that of the first oligonucleotide, and an oligonucleotide probe with a label and a third sequence that is complementary to the first and the second oligonucleotides. In a specific embodiment the oligosphere is magnetic. The document reports specific uses of oligospheres as references in standardization procedures.

Modified oligonucleotides such as peptide nucleic acid (PNA) and locked nucleic acid (LNA) have been explored for physiological applications. LNA possesses a methylene linker between the 2′-oxygen and 4′-carbon of the ribose moiety that consequently locks the sugar into a C3-endo conformation, hence the name “locked nucleic acid”. This chemical modification confers nuclease resistance as well as higher affinity and greater specificity for oligonucleotide targets in technical applications involving duplex formation by hybridization. WO 1998/39352 discloses locked nucleic acid (LNA) structures. WO 2000/056746 discloses synthesis of LNA monomers including intermediate products for certain stereoisomers of LNA. By way of chemical synthesis, single strands consisting of LNA nucleoside analog monomers only (“all-LNA”) can be synthesized.

As mentioned above, the locked nucleic acid (LNA) monomer is a conformationally restricted nucleotide analogue with an extra 2′-0,4′-C-methylene bridge added to the ribose ring. LNA monomers are provided as 2′-0,4′-C-methylene-(D-ribofuranosyl) nucleoside monomers (Singh S. K. et al. Chem. Commun. 4 (1998) 455-456; Koskin A. A. et al. Tetrahedron 54 (1998) 3607-3630; Wengel J. Acc. Chem. Res. 32 (1999) 301-310). WO2000/066604 and WO2000/056746 disclose certain stereoisomers of LNA nucleoside monomers.

Mixed DNA-LNA oligonucleotides that contain DNA and LNA monomers have shown stability towards 3′-exonucleolytic degradation and greatly enhanced thermal stability when hybridized to complementary DNA and RNA. In fact, in comparison with other high-affinity nucleic acid mimics that have been synthesized, e.g. peptide nucleic acids (PNAs), hexitol nucleic acids (HNAs) and 2′-fluoro N3′-phosphoramidates, LNA displays exceptional binding affinities. Hybridization kinetics of LNA-DNA mixed oligonucleotides, also known as “mixmers” were reported by Christensen U. et al. (Biochem J 354 (2001) 481-484). A crystal structure of a duplex from two complementary ss-oligonucleotides, each consisting of 7 LNA monomers was reported by Eichert A. et al. (Nucleic Acids Research 38 (2010) 6729-6736).

WO 1999/14226 suggests the use of LNA in the construction of affinity pairs for attachment to molecules of interest and solid supports. However it is also known to the art that hybridization of complementary all-LNA single strands poses technical problems. Thermodynamic analysis of all-LNA hybridization is largely empirical and sequence prediction of hybridizing monomers without a prior denaturation step (e.g. heating prior to hybridization) does not appear to be possible, so far.

For the most part, mixed LNA-DNA oligonucleotides (also referred to as “mixmer single strands” or “mixmers”) were analyzed, so far. Fewer reports of the characterization of hybridizing single-stranded oligonucleotides made exclusively from LNA monomers (i.e. “all-LNA” single-stranded oligonucleotides) were published, so far, particularly by Koshkin A. A. et al. (J Am Chem Soc 120 (1998) 13252-13253) and Mobile B. P. et al. (Analyst 130 (2005) 1634-1638). Eze N. A. et al. (Biomacromolecules 18 (2017) 1086-1096) report association rates from DNA-LNA mixmers and DNA probes to be below 10⁵ M⁻¹ s⁻¹. According to these authors, the hybridization kinetics in solution does not seem to be affected by substituting one or more DNA monomers with LNA monomers, considering one third of monomers available for substitution.

Predictions concerning thermodynamic behavior of LNA-containing oligonucleotides are aided by dedicated computer programs referred to by Tolstrup N et al. (Nucleic Acids Research 31 (2003) 3758-3762). However, this report explicitly mentions a higher prediction error for LNA oligonucleotides due to the more complex properties of these oligonucleotides, rather than lack of experimental data. Specifically, the present report demonstrates that complementary ss-oligonucleotides consisting of 8 or more LNA monomers are unpredictable with regards to their ability to form duplex molecules with Watson-Crick base pairing.

A general objective of the present report is therefore the identification and provision of binding pairs of single-stranded all-LNA oligonucleotides which are capable of hybridizing, thereby forming duplex molecules with Watson-Crick base pairing. More specifically, binding pairs are sought which are capable of duplex formation under non-denaturing conditions, more specifically under conditions which are compatible with the function of analyte-specific receptors in an analyte detection assay (such as, but not limited to, an immunoassay). Importantly, single-stranded all-LNA oligonucleotides are sought which can be stored and hybridized with each other in aqueous solution at ambient temperatures such as, but not limited to, room temperature, without an intermittent heating step to remove any intramolecular secondary structures which could cause hybridization incompatibility of the complementary oligonucleotides.

### SUMMARY OF THE INVENTION

The present disclosure, in a first aspect being related to all other aspects and embodiments as disclosed herein, provides a method for providing a binding pair, the binding pair consisting of a first single-stranded LNA oligonucleotide and a second single-stranded LNA oligonucleotide, the two oligonucleotides being capable of forming an antiparallel duplex of 8 to 15 consecutive Watson-Crick base pairs at a temperature from 20° C. to 40° C., the method comprising the steps of
(a) providing a first single-stranded (=ss-) oligonucleotide consisting of 8 to 15 locked nucleic acid (=LNA) monomers, each monomer comprising a nucleobase, the nucleobases of the first ss-oligonucleotide forming a first nucleobase sequence;
(b) providing a second ss-oligonucleotide consisting of 8 to 15 LNA monomers, the second ss-oligonucleotide consisting of at least the number of monomers as the first ss-oligonucleotide, each monomer of the second ss-oligonucleotide comprising a nucleobase, the nucleobases of the second ss-oligonucleotide forming a second nucleobase sequence of the second ss-oligonucleotide, the second nucleobase sequence comprising or consisting of a nucleobase sequence complementary to the first nucleobase sequence in antiparallel orientation and theoretically predicting the capability of the first and second ss-oligonucleotide to form a duplex with each other, the duplex consisting of 8 to 15 consecutive Watson-Crick base pairs;
(c) mixing and incubating for a time interval of 20 min or less in an aqueous solution at a temperature from 20° C. to 40° C. equal molar amounts of the first and second ss-oligonucleotides, thereby obtaining a mixture of ss-oligonucleotides or a duplex-containing mixture; followed by
(d) separating at a temperature from 20° C. to 40° C. the mixture obtained in step (c), followed by detecting and quantifying the separated duplex, and detecting and quantifying the separated ss-oligonucleotides; followed by
(e) selecting the binding pair if in step (d) duplex is detectably present, and if the molar amount of duplex is higher than the molar amount of ss-oligonucleotides;

thereby providing the binding pair.

The present disclosure, in a second aspect being related to all other aspects and embodiments as disclosed herein, provides a liquid composition comprising an aqueous solvent and a binding pair consisting of a first single-stranded oligonucleotide and a second single-stranded oligonucleotide,

wherein each oligonucleotide consists of 8 to 15 locked nucleic acid (=LNA) monomers, each monomer comprising a nucleobase, the nucleobases of the monomers forming a first nucleobase sequence of the first oligonucleotide and a second nucleobase sequence of the second oligonucleotide,

wherein the first nucleobase sequence and the second nucleobase sequence are selected that the first oligonucleotide and the second oligonucleotide are capable of forming an antiparallel duplex of 8 to 15 consecutive Watson-Crick base pairs at a temperature from 20° C. to 40° C.,

and wherein the binding pair is obtainable by a method according to the first aspect as disclosed herein.

### DETAILED DESCRIPTION OF THE INVENTION

A binding pair is understood as being a set of two different binding partners which under non-denaturing conditions, are capable of forming with each other a specific non-covalent intermolecular bond. In the context of the present disclosure, the broadest understanding of non-denaturing conditions denotes the absence of any externally applied influence, such as heating or the addition of an amount of a denaturating compound, to molecularly unfold the target substance, thereby disrupting its secondary or higher-order structure. In this regard, heating is exemplified by raising the temperature substantially above 40° C., 50° C., 60° C. or an even higher temperature for a desired time period, and a denaturing compound can be exemplified by a detergent, a chaotrope, or a compound capable of lowering the melting temperature of a nucleic acid duplex, such as formamide.

Importantly, each first and second binding partner does not form a specific bond with a partner of the same species. That is to say, a specific intramolecular bond between two first partners or two second partners does not occur. At the same time, under non-denaturing conditions each separate partner presents itself capable of binding the other partner. Specifically, under non-denaturing conditions the separate partner does not form any intramolecular bond which would render it incapable of forming a bond with a partner of the other species. E.g., intramolecular folding could lead to secondary structures which under non-denaturing conditions would be stable enough to inhibit or prevent the desired intermolecular bonding of the two different species of binding partners.

However, intramolecular folding affecting one or both binding partners might not necessarily completely inhibit the desired intermolecular bonding of the two different species; the kinetics of intermolecular bonding is expected to become slower compared to unimpeded binding partners without intramolecular folding. Particularly considering a standardized high-throughput assay setup such as (but not limited to) an automated immunoassay, such a setting typically requires fast formation of the intermolecularly connected form of the binding pair from the previously separate binding partners. Thus, absence of or largely minimized intramolecular folding in each binding partner is a desired technical feature.

In the context of the present disclosure, and with specific regard to immunoassays and the interaction of a receptor and its target substance (analyte), non-denaturing conditions are more specifically understood as the collective features of an environment which is permissive for the receptor (e.g. antibody) in attaining and/or maintaining the conformation which allows the receptor's interaction with and binding of its target substance (analyte). At the same time, the environment given by non-denaturing conditions is permissive for the target substance in attaining and/or maintaining the conformation which allows it to become and/or remain bound by the receptor.

Particularly all-LNA oligonucleotides have features which cannot be reliably predicted by the present tools that are available to the skilled person. For practical reasons, the present study was limited to ss-oligonucleotides consisting of up to 15 LNA monomers.

Thus, the present disclosure, in a first aspect being related to all other aspects and embodiments as disclosed herein, provides a method for providing a binding pair, the binding pair consisting of a first single-stranded LNA oligonucleotide and a second single-stranded LNA oligonucleotide, the two oligonucleotides being capable of forming an antiparallel duplex of 8 to 15 consecutive Watson-Crick base pairs at a temperature from 20° C. to 40° C., the method comprising the steps of
(a) providing a first single-stranded (=ss-) oligonucleotide consisting of 8 to 15 locked nucleic acid (=LNA) monomers, each monomer comprising a nucleobase, the nucleobases of the first ss-oligonucleotide forming a first nucleobase sequence;
(b) providing a second ss-oligonucleotide consisting of 8 to 15 LNA monomers, the second ss-oligonucleotide consisting of at least the number of monomers as the first ss-oligonucleotide, each monomer of the second ss-oligonucleotide comprising a nucleobase, the nucleobases of the second ss-oligonucleotide forming a second nucleobase sequence, the second nucleobase sequence comprising or consisting of a nucleobase sequence complementary to the first nucleobase sequence in antiparallel orientation and, by way of complementarity, predicting the capability of the first and second ss-oligonucleotide to form a duplex with each other, the duplex consisting of 8 to 15 consecutive Watson-Crick base pairs;
(c) mixing and incubating for a time interval of 20 min or less in an aqueous solution at a temperature from 20° C. to 40° C. equal molar amounts of the first and second ss-oligonucleotides, thereby obtaining a mixture of ss-oligonucleotides or a duplex-containing mixture; followed by
(d) separating at a temperature from 20° C. to 40° C. the mixture obtained in step (c), followed by detecting and quantifying the separated duplex, if present, and detecting and quantifying the separated ss-oligonucleotides; followed by
(e) selecting the binding pair if in step (d) duplex is detectably present, and if the molar amount of duplex is higher than the molar amount of ss-oligonucleotides;

thereby providing the binding pair.

An all-LNA ss-oligonucleotide as specified in here may contain a number of monomers, the number selected from the group consisting of 8, 9, 10, 11, 12, 13, 14, and 15. In an embodiment of all aspects and embodiments as disclosed herein, the first ss-oligonucleotide consists of 8 to 12 monomers (i.e. a number selected from 8, 9, 10, 11 and 12 monomers), and in a more specific embodiment of all aspects and embodiments as disclosed herein, the first ss-oligonucleotide consists of 8 monomers. In another embodiment of all aspects and embodiments as disclosed herein, the first ss-oligonucleotide consists of 8 to 10 monomers (i.e. a number selected from 8, 9, and 10 monomers), and in a more specific embodiment of all aspects and embodiments as disclosed herein, the first ss-oligonucleotide consists of 9 monomers. The first and the second ss-oligonucleotide do not need to be of equal size, i.e. need not consist of an equal number of monomers. However, an equal number of monomers making up the first and the second ss-oligonucleotide is a specific embodiment of all aspects and embodiments as disclosed herein.

Two oligonucleotides are antiparallel if they run parallel to each other but with opposite alignments. A specific example is the two complementary strands of a nucleic acid duplex, which run in opposite directions alongside each other. As a consequence, each end of the duplex comprises the 5′ end of the first strand next to/aligned with the 3′ end of the opposite second strand Similar to DNA and RNA, LNA exhibits Watson-Crick base pairing (Koshkin, A. A. et al. J Am Chem Soc 120 (1998) 13252-13260). In an embodiment of all aspects and embodiments as disclosed herein, each LNA monomer comprises a nucleobase selected from the group consisting of adenine, thymine, uracil, guanine, cytosine, 5-methylcytosine, 5-hydroxymethylcytosine, 7-deazaguanine and 7-deazaadenine. Specific Watson-Crick base pairing involving these bases on complementary opposite strands is an accepted feature well known to the skilled person and widely published in the art. Apart from the listed nucleobases others are known to the skilled person which can be incorporated in all-LNA ss-oligonucleotides, too. Generally, these include pyrimidines derivatized at the C-5 atom

Importantly, after contacting the two different (i.e. first and second) ss-oligonucleotides, step (c) of the method specifies incubation for a time interval of 20 min or less. In a specific embodiment of all aspects and embodiments as disclosed herein, the time interval is selected from the group consisting of 1 s to 20 min, 1 s to 15 min, 1 s to 10 min, 1 s to 5 min, 1 s to 1 min, 1 s to 30 s, 1 s to 20 s, 1 s to 10 s, and 1 s to 5 s. A very advantageous time interval is selected from 1 s to 10 s, and 1 s to 5 s.

In step (c) the temperature is selected independently from the temperature in step (d), and vice versa. In a specific embodiment of all aspects and embodiments as disclosed herein, the temperatures in step (c) and (d) do not differ by more than 5° C. In a specific embodiment of all aspects and embodiments as disclosed herein in step (c) and/or in step (d) the temperature is from 20° C. to 25° C. In a specific embodiment of all aspects and embodiments as disclosed herein in step (c) and/or in step (d) the temperature is from 25° C. to 37° C. In another specific embodiment of all aspects and embodiments as disclosed herein, prior to step (c) the first ss-oligonucleotide and the second ss-oligonucleotide are kept at a temperature from −80° C. to 40° C., specifically from 20° C. to 40° C., more specifically from 20° C. to 25° C., even more specifically from 25° C. to 37° C. In another embodiment of all aspects and embodiments as disclosed herein, in step (c) the aqueous solution contains a buffer maintaining the pH of the solution from pH 6 to pH 8, more specifically from pH 6.5 to pH 7.5. In another embodiment of all aspects and embodiments as disclosed herein, in step (c) the aqueous solution contains a salt. In another embodiment of all aspects and embodiments as disclosed herein, in step (c) the aqueous solution contains an aggregate amount of dissolved substances from 10 mmol/L to 500 mmol/L, more specifically from 200 mmol/L to 300 mmol/L, more specifically from 10 mmol/L to 150 mmol/L, more specifically from 50 mmol/L to 200 mmol/L.

In another embodiment of all aspects and embodiments as disclosed herein, step (d) comprises subjecting the incubated mixture of step (c) to column chromatography with an aqueous solvent as mobile phase. Thus, column chromatography is used to separate duplex molecules from ss-oligonucleotides. Suitable chromatography methods such as HPLC are well known to the skilled person in this regard.

In another embodiment of all aspects and embodiments as disclosed herein, the ss-oligonucleotides of (a) and (b) consist of beta-D-LNA monomers. That is to say, the first ss-oligonucleotide entirely consists of beta-D-LNA monomers, and the second ss-oligonucleotide entirely consists of beta-D-LNA monomers. In yet another embodiment of all aspects and embodiments as disclosed herein, the ss-oligonucleotides of (a) and (b) consist of beta-L-LNA monomers. That is to say, the first ss-oligonucleotide entirely consists of beta-L-LNA monomers, and the second ss-oligonucleotide entirely consists of beta-L-LNA monomers.

By way of the method as disclosed in there, and also by means of any of its embodiments, the present disclosure provides an all-LNA duplex formed at a pre-selected temperature from 25° C. to 40° C. from a non-denatured pair of complementary single-stranded all-LNA oligomers, each comprising from 8 to 15 LNA monomers.

The present disclosure, in a second aspect being related to all other aspects and embodiments as disclosed herein, provides a liquid composition comprising an aqueous solvent and a binding pair consisting of a first single-stranded oligonucleotide and a second single-stranded oligonucleotide, wherein each oligonucleotide consists of 8 to 15 locked nucleic acid (=LNA) monomers, each monomer comprising a nucleobase, the nucleobases of the monomers forming a first nucleobase sequence of the first oligonucleotide and a second nucleobase sequence of the second oligonucleotide, wherein the first nucleobase sequence and the second nucleobase sequence are selected that the first oligonucleotide and the second oligonucleotide are capable of forming an antiparallel duplex of 8 to 15 consecutive Watson-Crick base pairs at a temperature from 20° C. to 40° C., and wherein the binding pair is obtainable by a method according to the first aspect as disclosed herein.

In a specific embodiment of all aspects and embodiments as disclosed herein, there is provided a liquid composition comprising an aqueous solvent and a binding pair consisting of a first single-stranded oligonucleotide and a second single-stranded oligonucleotide, wherein each oligonucleotide consists of 8 to 15 locked nucleic acid (=LNA) monomers, each monomer comprising a nucleobase, the nucleobases of the monomers forming a first nucleobase sequence of the first oligonucleotide and a second nucleobase sequence of the second oligonucleotide, wherein the first nucleobase sequence and the second nucleobase sequence are selected that the first oligonucleotide and the second oligonucleotide are capable of forming an antiparallel duplex of 8 to 15 consecutive Watson-Crick base pairs at a temperature from 20° C. to 40° C., and wherein the binding pair is obtainable by a method according to the first aspect as disclosed herein.

In a specific embodiment of all aspects and embodiments as disclosed herein, there is provided a liquid composition comprising an aqueous solvent and a binding pair consisting of a first single-stranded oligonucleotide and a second single-stranded oligonucleotide, wherein each oligonucleotide consists of 8 to 15 locked nucleic acid (=LNA) monomers, each monomer comprising a nucleobase, the nucleobases of the monomers forming a first nucleobase sequence of the first oligonucleotide and a second nucleobase sequence of the second oligonucleotide, wherein the first nucleobase sequence and the second nucleobase sequence are selected that the first oligonucleotide and the second oligonucleotide are capable of forming an antiparallel duplex of 8 to 15 consecutive Watson-Crick base pairs at a temperature from 20° C. to 40° C., and wherein the binding pair is obtained by a method according to the first aspect as disclosed herein.

In an embodiment of all aspects and embodiments as disclosed herein, each oligonucleotide consists of 9 to 15 LNA monomers, wherein the first nucleobase sequence and the second nucleobase sequence are selected such that the first oligonucleotide and the second oligonucleotide are capable of forming an antiparallel duplex of 9 consecutive Watson-Crick base pairs at a temperature from 20° C. to 40° C., and wherein the binding pair is obtainable or obtained by a method according to a method of the first aspect and an embodiment thereof.

In an embodiment of all aspects and embodiments as disclosed herein, each ss-oligonucleotide contains two or three different nucleobases. In an embodiment of all aspects and embodiments as disclosed herein, the nucleobases in each ss-oligonucleotide the G+C (including analogs of G and C) content is lower than 75%. In a specific embodiment, the G+C content is lower than a value selected from 74%, 73%, 72%, 71%, and 70%. In yet another embodiment of all aspects and embodiments as disclosed herein, each LNA monomer in the binding pair comprises a nucleobase selected from the group consisting of adenine, thymine, uracil, guanine, cytosine, 5-methylcytosine, 5-hydroxymethylcytosine, 7-deazaguanine and 7-deazaadenine. In a more specific embodiment, among the nucleobases in each ss-oligonucleotide each cytosine is replaced by a 5-methylcytosine.

In an embodiment of all aspects and embodiments as disclosed herein, a binding pair of two separate compatible binding partners is a pair of all-LNA ss-oligonucleotides selected from the group consisting of

| | | | |
|---|---|---|---|
| 5′ tgctcctg 3′ | | and 5′ caggagca 3′, | |
| 5′ gcctgacg 3′ | | and 5′ cgtcaggc 3′, | |
| 5′ ctgcctgacg 3′ | (SEQ ID NO: 1) | and 5′ cgtcaggcag 3′, | (SEQ ID NO: 2) |
| 5′ gactgcctgacg 3′ | (SEQ ID NO: 3) | and 5′ cgtcaggcagtc 3′, | (SEQ ID NO: 4) |
| 5′ tgctcctgt 3′ | | and 5′ acaggagca 3′, | |
| 5′ gtgcgtct 3′ | | and 5′ agacgcac 3′, | |
| 5′ gttggtgt 3′ | | and 5′ acaccaac 3′, | |
| 5′ caacacaccaac 3′ | (SEQ ID NO: 5) | and 5′ gttggtgtgttg 3′, | (SEQ ID NO: 6) |
| 5′ acacaccaac 3′ | (SEQ ID NO: 7) | and 5′ gttggtgtgt 3′, | (SEQ ID NO: 8) |
| 5′ acaccaac 3′ | | and 5′ gttggtgt 3′ | |

In a specific embodiment, the monomers of the ss-oligonucleotides in any selected pair of the foregoing group are beta-D-LNA monomers. In yet another specific embodiment, the monomers of the ss-oligonucleotides in any selected pair of the foregoing group are beta-L-LNA monomers.

In an embodiment of all aspects and embodiments as disclosed herein, one ss-oligonucleotide of the binding pair is attached to a solid phase selected from the group consisting of magnetic bead, paramagnetic bead, synthetic organic polymer (latex) bead, polysaccharide bead, test tube, microwell plate cavity, cuvette, membrane, scaffolding molecule, quartz crystal, film, filter paper, disc and chip. In another embodiment of all aspects and embodiments as disclosed herein, one ss-oligonucleotide of the binding pair is connected to a molecule selected from the group consisting of peptide, polypeptide, oligonucleotide, polynucleotide, sugar, glycan, hapten, and dye. In yet another embodiment of all aspects and embodiments as disclosed herein, a ss-oligonucleotide is attached covalently to a linker. In yet another embodiment of all aspects and embodiments as disclosed herein, a ss-oligonucleotide is attached covalently to an analyte-specific receptor useful in a receptor-based analyte detection assay such as, but not limited to, an immunoassay.

In its broadest sense and in line with the generally accepted understanding in biochemistry, a receptor is a structure which has an affinity for a specific target molecule as a whole, or an affinity for a specific molecular region and/or three-dimensional aspect of the target molecule. For the purpose of the present disclosure, a receptor is understood to interact with and bind a target molecule. In biochemical assays a receptor can be used to capture its target molecule to separate the target from a complex mixture, and to determine the target molecule as an analyte. By way of example, immunoassays typically use antibodies or antibody-derived molecules as receptors. A capture receptor is a receptor which is either provided in immobilized form (i.e. attached to a solid phase), or, preferred, in a form which is capable of being immobilized Immobilization can be effected by means of a binding pair connecting, or capable of connecting, the solid phase and the receptor.

In very general terms, an immunoassay provides one or more receptors which are capable of specifically binding to a target analyte. Such receptors can be exemplified by analyte-specific immunoglobulins; hence the name immunoassay. However, for the purpose of the present disclosure, any other type of analyte-specific receptor is considered, too. Thus, the more general term receptor-based analyte detection assay is appropriate.

Typically, the target analyte is comprised in a sample, wherein the sample is a complex mixture of different molecules. For the purpose of the present disclosure, a liquid sample is considered. The liquid sample comprises a liquid phase, i.e. a liquid solvent which usually is an aqueous solvent. In the aqueous solvent a plurality of molecules are present in dissolved state. Thus, in a specific embodiment the sample is in a liquid state of aggregation, and it is a monophasic homogeneous mixture. In a specific embodiment the analyte is comprised in the mixture in dissolved form, and in addition one or more further molecules are present in the mixture in dissolved form.

With regards to detection, by way of receptor-based analyte detection assay, of a target analyte which is present in the liquid sample, or which is suspected to be present therein, in a first essential step, the analyte is specifically bound. Specific binding implies that a receptor is or becomes present, wherein the receptor has a binding affinity and binding specificity for the analyte which are high for the target analyte and low or absent for the further molecules which are also present in the sample. In a specific embodiment (and exemplifying a large number of existing assays), a compound comprising a receptor capable of specifically binding to the analyte is added to the sample. Importantly, the mixture of the sample and the compound comprising the receptor must provide conditions which are permissive to the specific interaction of the receptor and the target analyte in the sample. This includes that in the mixture the conditions must be permissive to the actual binding of the analyte by the receptor, and they are desired to stabilize the receptor with the bound target analyte. At the same time, the mixture of the sample and compound is desired not to favor or stabilize unspecific binding of further molecules to the receptor, or to the compound comprising the receptor as a whole.

Subsequently, the analyte is immobilized Immobilization is an important step in the detection process as it allows to separate the analyte from the surrounding complex mixture, specifically from the further molecules of the sample. Immobilization requires a solid phase to which the target analyte becomes attached. Once immobilized, the analyte can be separated from the mixture by way of phase separation. Separated from the mixture (i.e. purified) the analyte is then detected.

Considering a receptor-based analyte detection assay and the immobilization step there is the need to provide a solid phase and to build a connection between the solid phase and the target analyte. It is desired that the connection builds up in a self-assembly process.

Immunoassays are well-established bioanalytical methods in which detection or quantitation of an analyte depends on the reaction of the analyte and at least one analyte-specific receptor, thus forming an analyte:receptor complex. A non-limiting example is the reaction between an antigen and an antibody, respectively. The specific embodiment of a “sandwich” immunoassay can be used for analytes possessing more than one recognition epitopes. Thus, a sandwich assay requires at least two receptors that attach to non-overlapping epitopes on the analyte. In a “heterogeneous sandwich immunoassay” one of the receptors has the functional role of an analyte-specific capture receptor; this receptor is or (during the course of the assay) becomes immobilized on a solid phase. A second analyte-specific receptor is supplied in dissolved form in the liquid phase. A sandwich-like complex is formed once the respective analyte is bound by a first and a second receptor (receptor-1:analyte:receptor-2). The sandwich-like complex is also referred to as “detection complex”. Within the detection complex the analyte is sandwiched between the receptors, i.e. in such a complex the analyte represents a connecting element between the first receptor and a second receptor.

The term “heterogeneous” (as opposed to “homogeneous”) denotes two essential and separate steps in the assay procedure. In the first step a detection complex containing label is formed and immobilized, however with unbound label still surrounding the complexes. Prior to determination of a label-dependent signal unbound label is washed away from immobilized detection complex, thus representing the second step. In contrast, a homogeneous assay produces an analyte-dependent detectable signal by way of single-step incubation and does not require a washing step.

In a heterogeneous assay the solid phase is functionalized such that it may have bound to its surface the functional capture receptor (the first receptor), prior to being contacted with the analyte; or the surface of the solid phase is functionalized in order to be capable of anchoring a first receptor, after it has reacted with the analyte. In the latter case the anchoring process must not interfere with the receptor's ability to specifically capture and bind the analyte. A second receptor present in the liquid phase is used for detection of bound analyte. Thus, in a heterogeneous immunoassay the analyte is allowed to bind to the first (capture) and second (detector) receptors. Thereby a “detection complex” is formed wherein the analyte is sandwiched between the capture receptor and the detector receptor. In a typical embodiment the detector receptor is labeled prior to being contacted with the analyte; alternatively a label is specifically attached to the detector receptor after analyte binding. With the detection complexes being immobilized on the solid phase the amount of label detectable on the solid phase corresponds to the amount of sandwiched analyte. After removal of unbound label with a washing step, immobilized label indicating presence and amount of analyte can be detected.

Another well-known embodiment is a competitive heterogeneous immunoassay which in its simplest form differs from the sandwich-type format by the lack of a second detector receptor. In contrast, the sample with the analyte is mixed with an artificially produced labeled analogon that is capable of cross-reacting with the analyte-specific receptor. In the assay the analyte and the analogon compete for binding to a capture receptor which is or becomes immobilized. Following the binding step, the higher the amount of immobilized label, the smaller the amount of the non-labeled analyte that was capable of competing for the capture receptor. Immobilized label is determined after a washing step. So the amount of label that is detectable on the solid phase inversely corresponds to the amount of analyte that was initially present in the sample.

Any washing step(s) necessary in a heterogeneous immunoassay require(s) the non-covalent connection of the first binding partner and the second binding partner to be sufficiently stable. However, the extent of required stability of the connection depends on the strength of the washing step(s) to be applied. Importantly and unexpectedly, a binding pair as demonstrated herein is exceptionally well suited to facilitate the immobilization step in an immunoassay. That is to say, in an immunoassay e.g. a first binding partner of the binding pair attached to a solid phase, and a second binding partner of the binding pair attached to an analyte-specific (capture) receptor are well suited to facilitate immobilization of the receptor on the solid phase.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 HPLC analysis of single-stranded LNA 1 (Example 2)

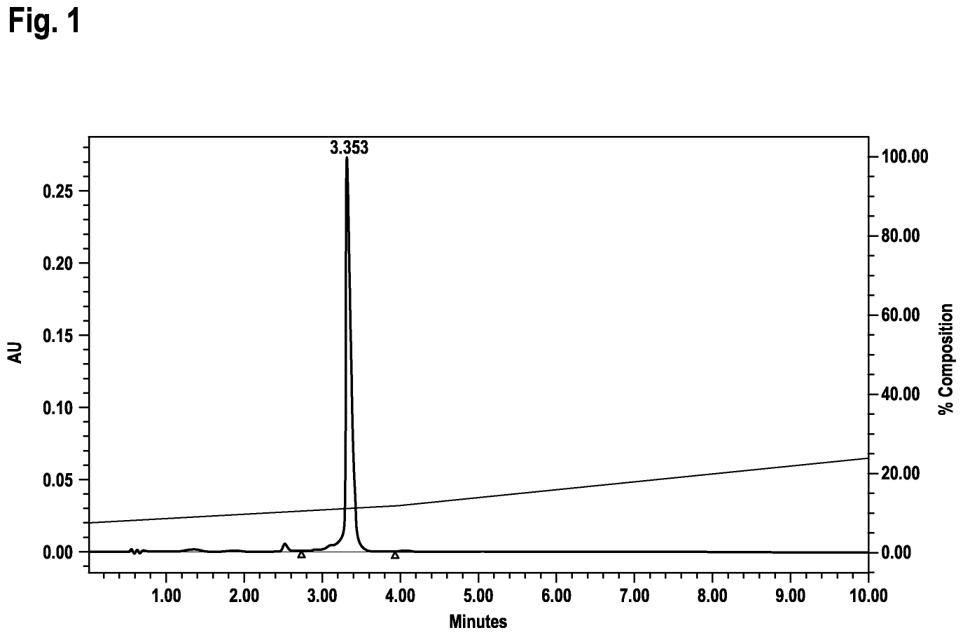

FIG. 2 HPLC analysis of single-stranded LNA 2 (Example 2)

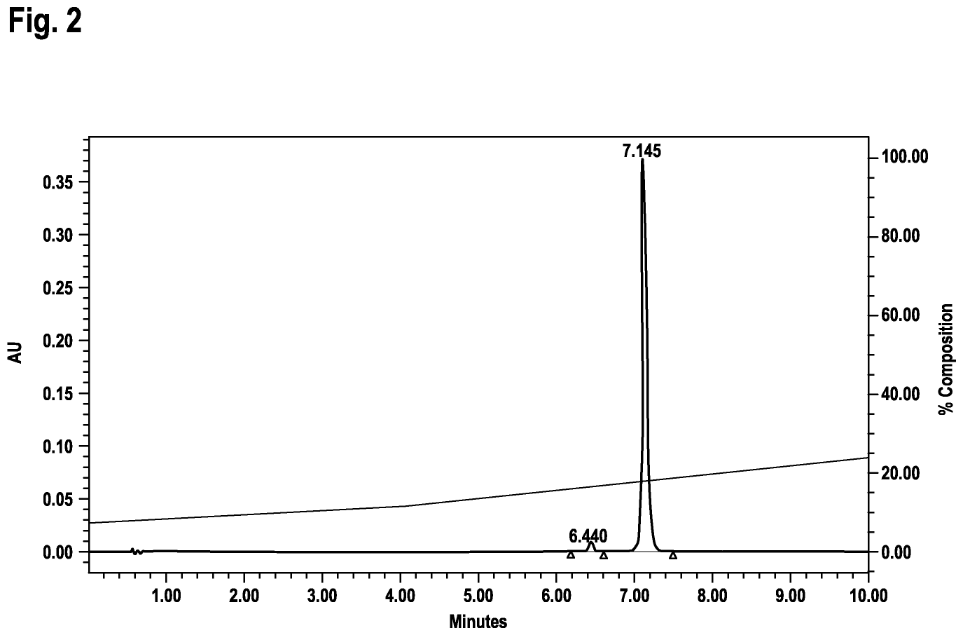

FIG. 3 HPLC analysis of mixed LNA 1 and LNA 2, immediate injection into HPLC system (Example 2)

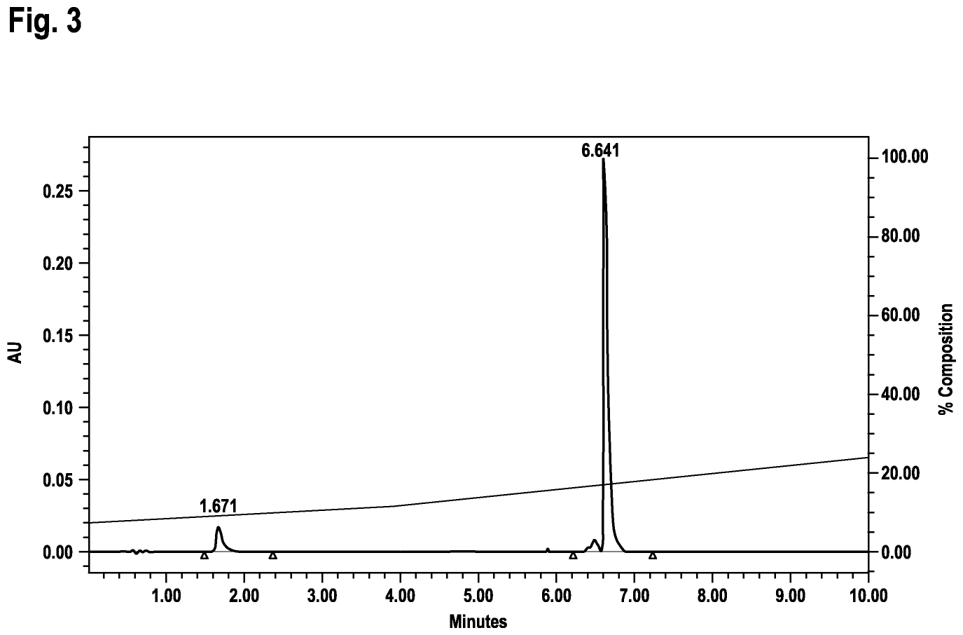

FIG. 4 HPLC analysis of mixed LNA 1 and LNA 2 after thermal denaturation prior to injection (Example 2) positive control: duplex formation

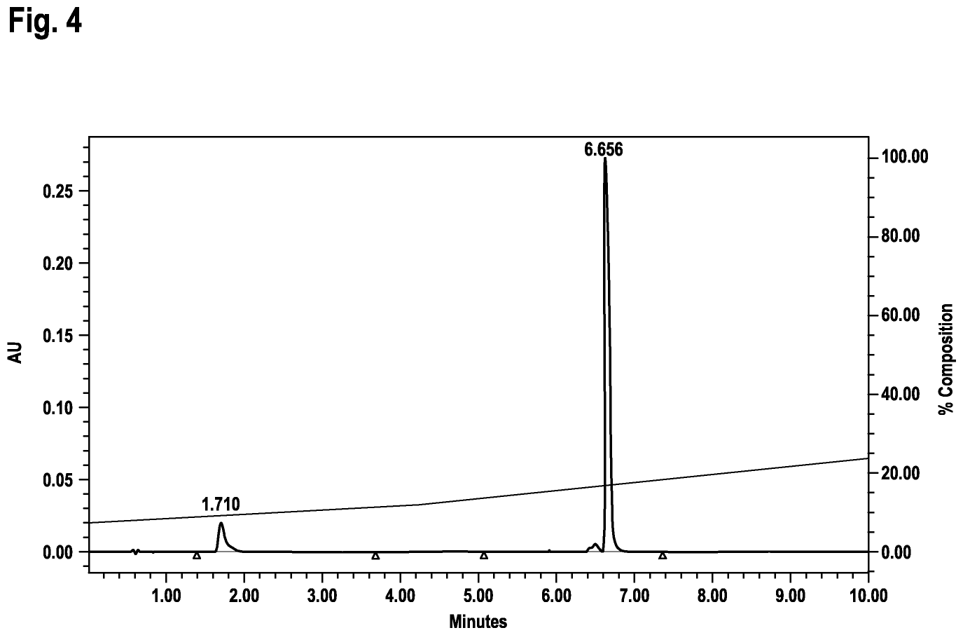

FIG. 5 HPLC analysis of single-stranded LNA 3 (Example 2)

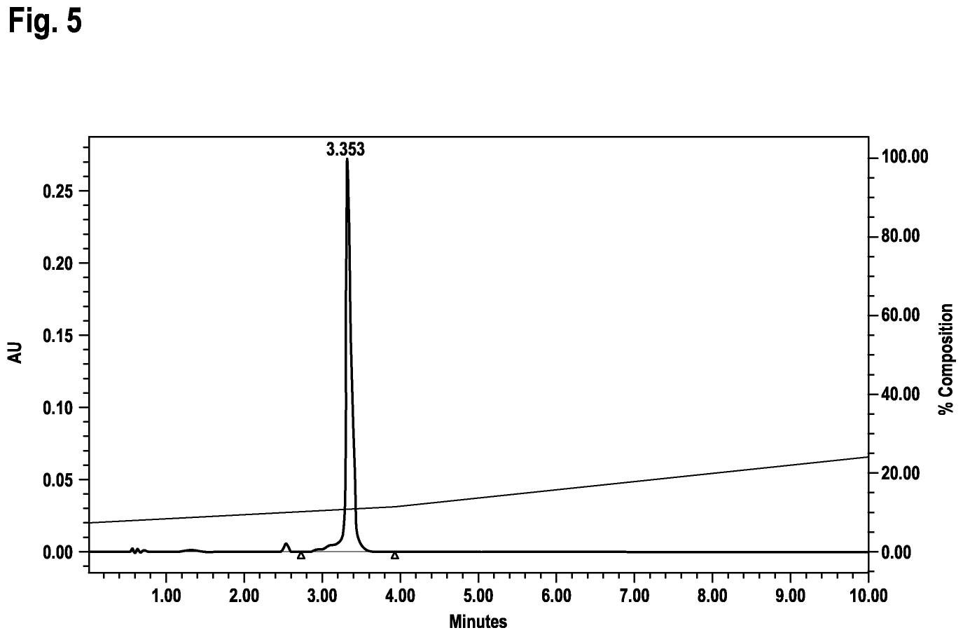

FIG. 6 HPLC analysis of single-stranded LNA 4 (Example 2)

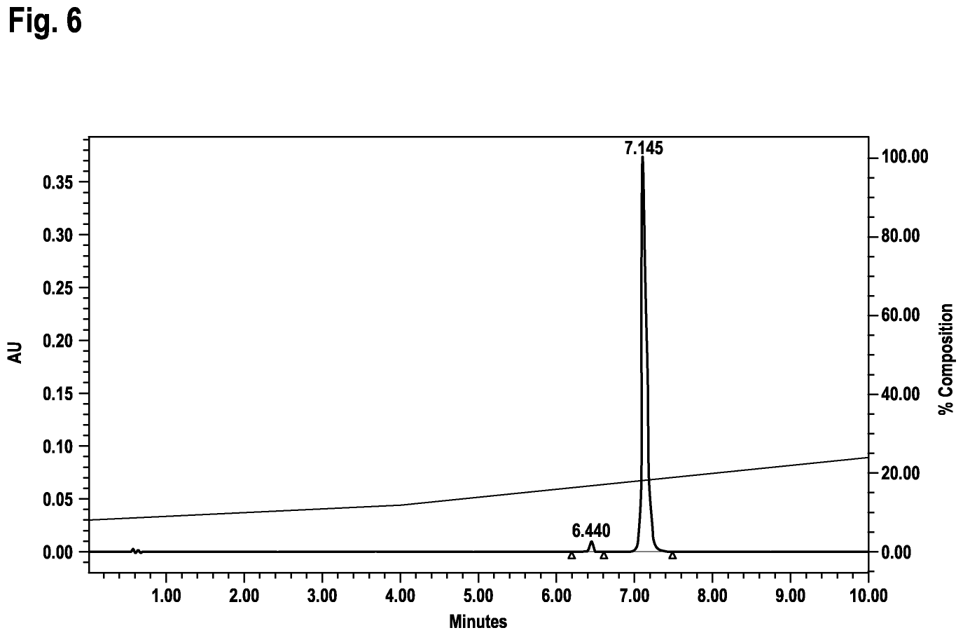

FIG. 7 HPLC analysis of mixed LNA 3 and LNA 4, immediate injection into HPLC system (Example 2) slow duplex formation (ratio<0.05)

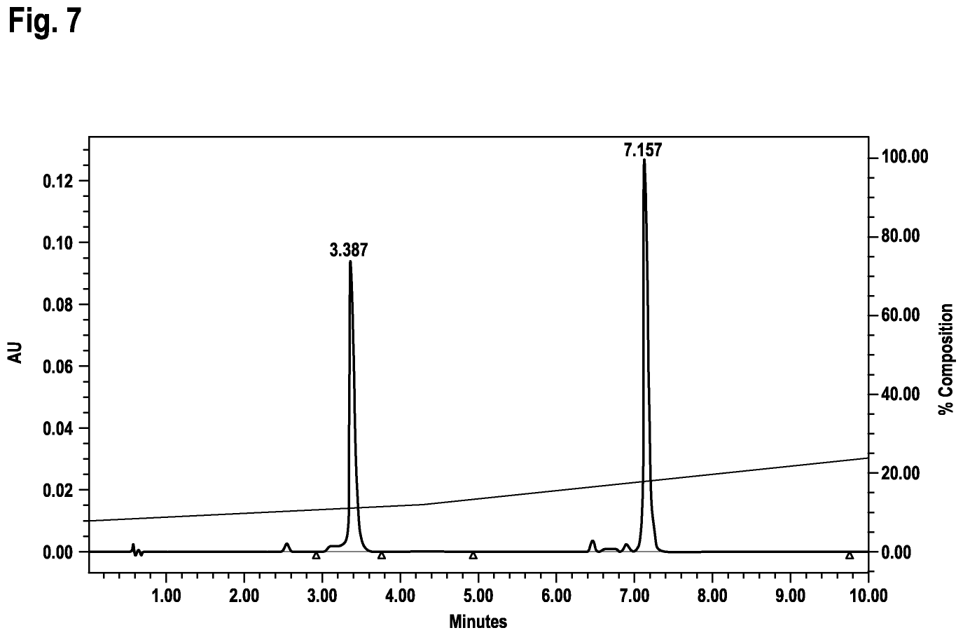

FIG. 8 HPLC analysis of mixed LNA 3 and LNA 4, injection after 50 min (Example 2) slow duplex formation (ratio=0.05)

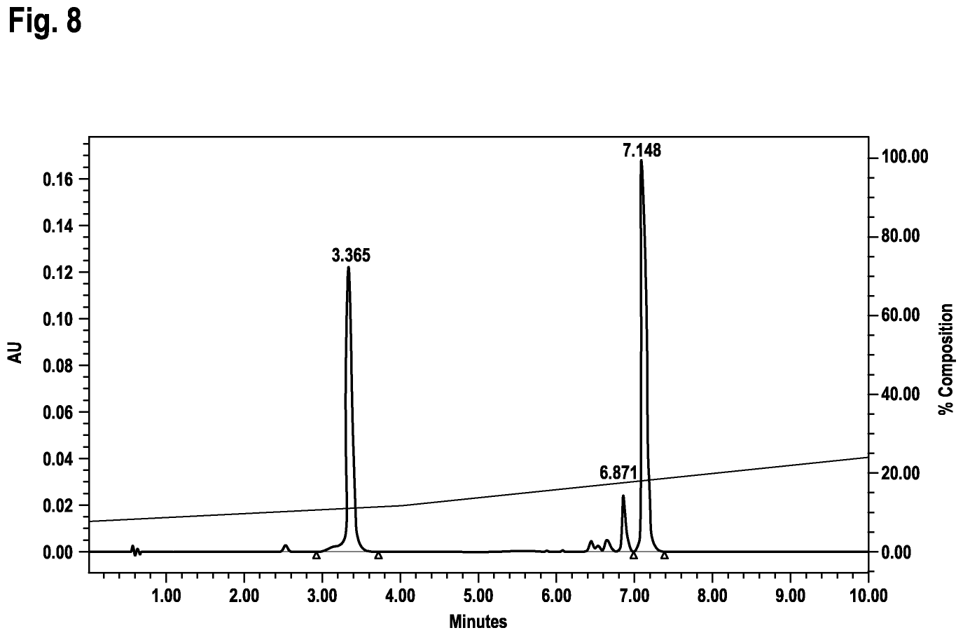

FIG. 9 HPLC analysis of mixed LNA 3 and LNA 4 after thermal denaturation prior to injection (Example 2) positive control: duplex formation

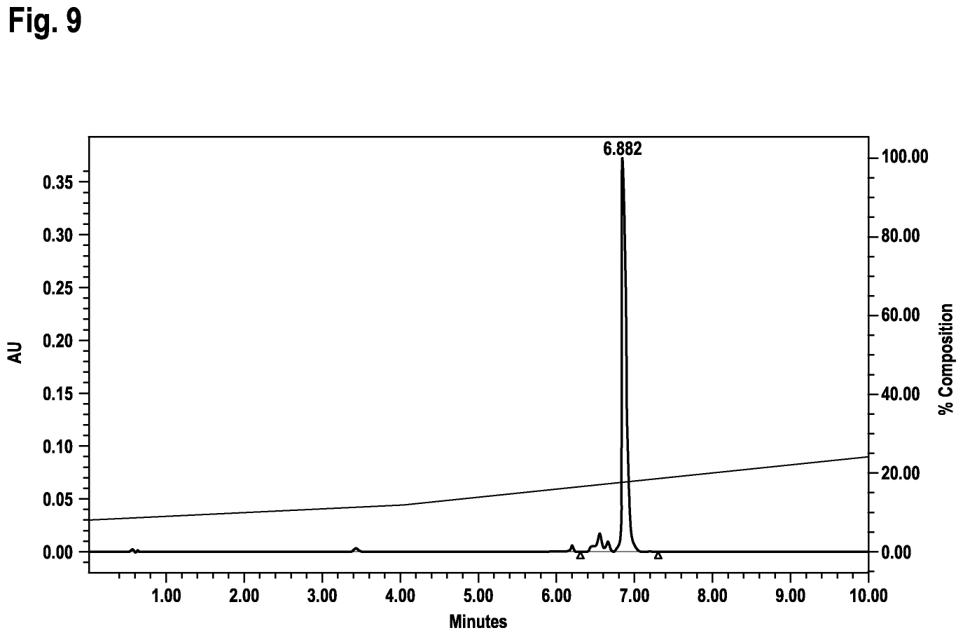

### EXAMPLE 1

Synthesis of LNA Oligonucleotides

LNA oligonucleotides were synthesized in a 1 μmole scale synthesis on an ABI 394 DNA synthesizer using standard automated solid phase DNA synthesis procedure and applying phosphoramidite chemistry. Glen UnySupport PS (Glen Research cat no. 26-5040) and LNA phosphoramidites (Qiagen/Exiqon cat. No. 33970 (LNA-A(Bz), 339702 (LNA-T), 339705 (LNA-mC(Bz) and 339706 (LNA-G(dmf); ß-L-LNA analogues were synthesized analogously to ß-D-LNA phosphoramidites starting from L-glucose (Carbosynth, cat. No. MG05247) according to A. A. Koshkin et al., J. Org. Chem 2001, 66, 8504-8512) as well as spacer phosphoramidte 18 (Glen Research cat. No. 10-1918) and 5′-Biotin phosphoramidte (Glen Research cat. No. 10-5950) were used as building blocks. All phosphoramidites were applied at a concentration of 0.1 M in DNA grade acetonitrile. Standard DNA cycles with extended coupling time (180 sec), extended oxidation (45 sec) and detritylation time (85 sec) and standard synthesis reagents and solvents were used for the assembly of the LNA oligonucleotides. 5′-biotinylated LNA oligonucleotides were synthesized DMToff, whereas unmodified LNA oligonucleotides were synthesized as DMTon. Then, a standard cleavage program was applied for the cleavage of the LNA oligonucleotides from the support by conc. ammonia. Residual protecting groups were cleaved by treatment with conc ammonia (8 h at 56° C.). Crude LNA oligonucleotides were evaporated and purified by RP HPLC (column: PRP-1, 7 μm, 250×21.5 mm (Hamilton, part no. 79352) or XBridge BEH C18 OBD, 5 μm, 10×250 mm (Waters part no. 186008167) using a 0.1 M triethylammonium acetate pH 7/acetonitrile gradient. Product fractions were combined and desalted by dialysis (MWCO 1000, SpectraPor 6, part no. 132638) against water for 3 days, thereby also cleaving DMT group of DMTon purified oligonucleotides. Finally, the LNA oligonucleotides were lyophilized.

Yields ranged from 85 to 360 nmoles.

LNA oligonucleotides were analyzed by RP18 HPLC (Chromolith RP18e, Merck part no. 1.02129.0001) using a 0.1 M triethylammonium acetate pH 7/acetonitrile gradient. Typical purities were >90%. Identity of LNA oligonucleotides were confirmed by LC-MS analysis.

### EXAMPLE 2

Identification of LNA Oligonucleotide Sequences Capable of Forming Duplex without Prior Denaturation Applying RP-HPLC Analysis

a) General Method:

LNA oligonucleotides from example 1 were dissolved in buffer (0.01 M Hepes pH 7.4, 0.15 M NaCl) and analyzed on RP18 HPLC (Chromolith RP18e, Merck part no. 1.02129.0001) using a 0.1 M triethylammonium acetate pH 7/acetonitrile gradient (8-24% acetonitrile in 10 min; detection at 260 nm).

Strand and corresponding counterstrand LNA oligonucleotides were mixed at equimolar concentration at r.t. (room temperature) and immediately analyzed on RP18 HPLC (Chromolith RP18e, Merck part no. 1.02129.0001) using a 0.1 M triethylammonium acetate pH 7/acetonitrile gradient (8-24% B in 10 min; detection at 260 nm).

In a first control experiment strand and corresponding counterstrand LNA oligonucleotides were mixed at equimolar concentration at r.t., incubated 1 h at r.t.

and thereafter analyzed on RP18 HPLC (Chromolith RP18e, Merck part no. 1.02129.0001) using a 0.1 M triethylammonium acetate pH 7/acetonitrile gradient (8-25% acetonitrile in 10 min; detection at 260 nm).

In a second control experiment to show duplex formation (positive control) strand and corresponding counterstrand LNA oligonucleotides were mixed at equimolar concentration at r.t., thermally denaturated at 95° C. (10 min), and after having reached r.t. again analyzed on RP18 HPLC (Chromolith RP18e, Merck part no. 1.02129.0001) using a 0.1 M triethylammonium acetate pH 7/acetonitrile gradient (8-24% acetonitrile in 10 min; detection at 260 nm).

Duplex formation can be detected if new peak at different retention time compared to the individual single stranded LNA oligonucleotides is formed. In the positive control mixed strand and counterstrand are thermally denaturated prior to injection yielding duplex. By time dependent injection after mixing strand and counterstrand LNA at r.t. without prior denaturation kinetics of duplex formation can be monitored.

LNA sequences are determined to be capable of quickly forming duplex if the HPLC % ratio of formed duplex and one of both single stranded LNA (corrected by extinction coefficient; in case both strands are not exactly equimolar higher ratio value is considered) is >0.9 after tempering 5-60 min at r.t. without prior denaturation (HPLC % corrected by extinction coefficients; hyperchromicity of duplex not considered).

b) Identification of Sequence which Forms Duplex Fast

LNA 1:
5′-tgctcctg-3'
LNA 2:
5′-Bi-Heg-caggagca-3′

Heg=hexaethyleneglycol

Bi=biotin label attached via the carboxy function of the valeric acid moiety of biotin

The results are displayed in Figures.

c) Identification of Sequence which Forms Duplex Slowly

LNA 3:
(SEQ ID NO: 1)
5′-ctgcctgacg-3′
LNA 4:
(SEQ ID NO: 2)
5'-Bi-Heg-cgtcaggcag-3′

The results are displayed in Figures.

calculation of ratio:

| | retention | | extinction | |
|---|---|---|---|---|
| | time | | coefficient (ε) | HPLC % |
| LNA | [min] | HPLC % | [l*mol⁻¹*cm⁻¹] | * ε⁻¹ * 1000 |
| LNA 3 single | 3.365 | 45.14 | 98900 | 0.456 |
| strand | | | | |
| LNA 4 single | 7.148 | 49.98 | 109300 | 0.457 |
| strand | | | | |
| LNA 3/LNA 4 | 6.871 | 4.88 | 208200 | 0.023 |
| double strand | | | | |

HPLC % * ε⁻¹ * 1000 (LNA 3/LNA 4 double strand) / HPLC % * ε⁻¹ * 1000 (LNA 3 single strand) = 0.023/0.456 = 0.05
HPLC % * ε⁻¹ * 1000 (LNA 3/LNA 4 double strand) / HPLC % * ε⁻¹ * 1000 (LNA 4 single strand) = 0.023/0.457 = 0.05

What is claimed is:

1. A method for providing a binding pair, the binding pair consisting of a first single-stranded (ss) locked nucleic acid (LNA) oligonucleotide and a second single-stranded LNA oligonucleotide, the first ss-LNA oligonucleotide and the second ss-LNA oligonucleotide being capable of forming an antiparallel duplex of 8 to 15 consecutive Watson-Crick base pairs at a temperature from about 20° C. to about 40° C., the method comprising the steps of:
(a) providing a first ss-LNA oligonucleotide consisting of 8 to 15 LNA monomers, each LNA monomer comprising a nucleobase, the nucleobases of the first ss-LNA oligonucleotide forming a first nucleobase sequence of the first ss-LNA oligonucleotide;
(b) providing a second ss-LNA oligonucleotide consisting of 8 to 15 LNA monomers, the second ss-LNA oligonucleotide consisting of at least the same number of LNA monomers as the first ss-LNA oligonucleotide, each LNA monomer of the second ss-LNA oligonucleotide comprising a nucleobase, the nucleobases of the second ss-LNA oligonucleotide forming a second nucleobase sequence of the second ss-LNA oligonucleotide, the second nucleobase sequence comprising a nucleobase sequence complementary to the first nucleobase sequence in antiparallel orientation, wherein the first ss-LNA oligonucleotide and the second ss-LNA oligonucleotide have the capability to form an antiparallel duplex with each other, the antiparallel duplex consisting of 8 to 15 consecutive Watson-Crick base pairs;
(c) mixing equal molar amounts of the first ss-LNA oligonucleotide and the second ss-LNA oligonucleotide in an aqueous solution to obtain a mixture and incubating the mixture for a time interval of 20 minutes or less at a temperature ranging from about 20° C. to about 40° C. to form the antiparallel duplex;
(d) separating the antiparallel duplex, if present, the first ss-LNA oligonucleotides and the second ss-LNA oligonucleotides from the mixture in step (c) at a temperature ranging from about 20° C. to about 40° C., followed by detecting and quantifying the separated antiparallel duplex, the separated first ss-LNA oligonucleotides and the separated second ss-LNA oligonucleotides;
(e) selecting the separated antiparallel duplex as the binding pair if in step (d) the antiparallel duplex is detectably present, and if the molar amount of the antiparallel duplex is higher than the molar amounts of the separated first ss-LNA oligonucleotides and the separated second ss-LNA oligonucleotides; thereby providing the binding pair.

2. The method according to claim 1, wherein the first ss-LNA oligonucleotide consists of 8 to 12 LNA monomers.

3. The method according to claim 2, wherein the first ss-LNA oligonucleotide consists of 9 LNA monomers.

4. The method according to claim 1, wherein each LNA monomer comprises a nucleobase selected from the group consisting of adenine, thymine, uracil, guanine, cytosine, 5-methylcytosine, 5-hydroxymethylcytosine, 7-deazaguanine and 7-deazaadenine.

5. The method according to claim 1, wherein in step (c) the temperature ranges from about 20° C. to about 37° C.

6. The method according to claim 1, wherein prior to step (c) the first ss-LNA oligonucleotide and the second ss-LNA oligonucleotide are kept at a temperature ranging from about −80° C. to about 40° C.

7. The method according to claim 1, wherein in step (c) the incubation is performed for 1 minute or less.

8. The method according to claim 1, wherein in step (c) the aqueous solution contains a buffer maintaining the pH of the solution from about pH 6 to about pH 8.

9. The method according to claim 1, wherein in step (c) the aqueous solution contains an aggregate amount of dissolved substances from 10 mmol/L to 500 mmol/L.

10. The method according to claim 1, wherein step (d) comprises subjecting the incubated mixture of step (c) to column chromatography with an aqueous solvent as mobile phase.

11. The method according to claim 1, wherein the first ss-LNA oligonucleotides and the second ss-LNA oligonucleotides of (a) and (b) consist of beta-D-LNA monomers.

12. The method according to claim 1, wherein the first ss-LNA oligonucleotides and the second ss-LNA oligonucleotides of (a) and (b) consist of beta-L-LNA monomers.

13. A liquid composition comprising an aqueous solvent and a binding pair, the binding pair comprising a first single-stranded (ss-) locked nucleic acid (LNA) oligonucleotide and a second ss-LNA oligonucleotide, wherein each of the first ss-LNA oligonucleotide and the second ss-LNA oligonucleotide consists of 8 to 15 LNA monomers, each LNA monomer comprising a nucleobase, the nucleobases of the LNA monomers forming a first nucleobase sequence of the first ss-LNA oligonucleotide and a second nucleobase sequence of the second ss-LNA oligonucleotide, and
wherein the first ss-LNA oligonucleotide and the second ss-LNA oligonucleotide form an antiparallel duplex of 8 to 15 consecutive Watson-Crick base pairs at a temperature from 20° C. to 40° C.

14. The composition according to item 13, wherein each of the first ss-LNA oligonucleotide and the second ss-LNA oligonucleotide consists of 8 to 15 LNA monomers, and wherein the first ss-LNA oligonucleotide and the second ss-LNA oligonucleotide form an antiparallel duplex of 8 to 12 consecutive Watson-Crick base pairs at a temperature from 20° C. to 40° C.

15. The composition according to claim 14, wherein each of the first ss-LNA oligonucleotide and the second ss-LNA oligonucleotide consists of 8 to 15 LNA monomers, and wherein the first ss-LNA oligonucleotide and the second ss-LNA oligonucleotide form an antiparallel duplex of 9 consecutive Watson-Crick base pairs at a temperature from 20° C. to 40° C.

16. The composition according to claim 13, wherein each LNA monomer comprises a nucleobase selected from the group consisting of adenine, thymine, uracil, guanine, cytosine, and 5-methylcytosine.

17. The composition according to claim 13, wherein each of the first ss-LNA oligonucleotide and the second ss-LNA oligonucleotide contains two or three different nucleobases.

18. The composition according to claim 17, wherein among the nucleobases in each of the first ss-LNA oligonucleotide and the second ss-LNA oligonucleotide the G+C content is lower than 75%.

19. The composition according to claim 17, wherein among the nucleobases in each of the first ss-LNA oligonucleotide and the second ss-LNA oligonucleotide each cytosine is replaced by a 5-methylcytosine.

20. A kit for performing a heterogeneous immunoassay for detecting an analyte, the kit containing in separate containers a solid phase having attached thereto the first ss-LNA oligonucleotide of the binding pair according to claim 13, and an analyte-specific receptor having attached thereto the second ss-LNA oligonucleotide of the binding pair according to claim 13.


SEQUENCE LISTING (8 sequences)

SEQ ID NO: 1 - DNA, 10 bp, Artificial Sequence
  Synthetic
ctgcctgacg

SEQ ID NO: 2 - DNA, 10 bp, Artificial Sequence
  Synthetic
cgtcaggcag

SEQ ID NO: 3 - DNA, 12 bp, Artificial Sequence
  Synthetic
gactgcctgacg

SEQ ID NO: 4 - DNA, 12 bp, Artificial Sequence
  Synthetic
cgtcaggcagtc

SEQ ID NO: 5 - DNA, 12 bp, Artificial Sequence
  Synthetic
caacacaccaac

SEQ ID NO: 6 - DNA, 12 bp, Artificial Sequence
  Synthetic
gttggtgtgttg

SEQ ID NO: 7 - DNA, 10 bp, Artificial Sequence
  Synthetic
acacaccaac

SEQ ID NO: 8 - DNA, 10 bp, Artificial Sequence
  Synthetic
gttggtgtgt